(19) United States
(12) Patent Application Publication
(10) Pub. No.: US 2020/0205740 A1
(43) Pub. Date: Jul. 2, 2020

(54) REAL-TIME ANALYSIS OF INPUT TO MACHINE LEARNING MODELS

(71) Applicant: X Development LLC, Mountain View, CA (US)

(72) Inventors: Sarah Ann Laszlo, Mountain View, CA (US); Aysja Johnson, Berkeley, CA (US); Georgios Evangelopoulos, Venice, CA (US); Nina Thigpen, Sunnyvale, CA (US); Yvonne Yip, San Francisco, CA (US)

(21) Appl. No.: 16/284,561

(22) Filed: Feb. 25, 2019

(30) Foreign Application Priority Data
Dec. 28, 2018 (GR) 20180100571

(51) Int. Cl. A61B 5/00; G06N 3/08; A61B 5/04; A61B 5/0478

(52) U.S. Cl. CPC A61B 5/7267; G06N 3/08; A61B 5/6803; A61B 5/0478; A61B 5/04012

(57) ABSTRACT

Methods, systems, and apparatus, including computer programs encoded on a computer storage medium, for obtaining feature sets for a first number of diagnostic trials performed with a patient for diagnostic testing, wherein each feature set includes one or more features of electroencephalogram (EEG) signals measured from the patient while the patient is presented with trial content known to stimulate one or more desired human brain systems. Iteratively providing different combinations of the feature sets as input data to a diagnostic machine learning model to obtain model outputs, each model output corresponding to a particular one of the combinations. Determining, based on the model outputs, a consistency metric, the consistency metric indicating whether a quantity of feature sets in the combinations is sufficient to produce accurate output from the diagnostic machine learning model. Selectively ending the diagnostic testing with the patient based on a value of the consistency metric.

### CROSS-REFERENCE TO RELATED APPLICATION

This application claims priority to Greek Patent Application No. 20180100571, filed on Dec. 28, 2018, entitled “REAL-TIME ANALYSIS OF INPUT TO MACHINE LEARNING MODELS,” the entirety of which is hereby incorporated by reference.

### TECHNICAL FIELD

This disclosure generally relates to machine learning systems. More particularly the disclosure relates to processes for improving the data input process for diagnostic machine learning systems.

### BACKGROUND

Processes of gathering data as input for diagnostic systems can be tedious, expensive, and invasive. This is especially the case when the subject is a human patient. Often, diagnostic systems are designed to gather much more data than required to produce an accurate diagnostic output. Such systems err on the side of gathering excess data rather than risk gathering an insufficient quantity of data that provides a less accurate output.

### SUMMARY

In general, the disclosure relates to a machine learning system that is configured to analyze the sufficiency of input data to produce accurate model outputs as the data is received by the system. More specifically, disclosure relates to processes that evaluate the performance of a diagnostic machine learning model as input is being gathered so that the input gathering process can be concluded once a sufficient amount of model input is gathered to ensure an accurate model prediction. For example, diagnostic machine learning models may perform real-time tests or measurements of behaviors of a human patient or operations of a test subject in order to diagnose a condition of the patient or subject. Implementations iteratively run the model on the measured input data as the data is being collected and monitor the performance (e.g., consistency) of the model's outputs in response to the data. The testing or measurements on the patient or subject can be ended as soon as the system determines sufficient data has been gathered to produce reliable and accurate model outputs. Diagnostic machine learning models can include, but are not limited to, medical diagnostic models, psychiatric diagnostic models, software or hardware diagnostic systems, or any other model that gathers or measures input data in real-time.

In general, innovative aspects of the subject matter described in this specification can be embodied in methods that include the actions of obtaining feature sets for a first number of diagnostic trials performed with a patient for diagnostic testing, wherein each feature set includes one or more features of electroencephalogram (EEG) signals measured from the patient while the patient is presented with trial content known to stimulate one or more desired human brain systems. Iteratively providing different combinations of the feature sets as input data to a diagnostic machine learning model to obtain model outputs, each model output corresponding to a particular one of the combinations. Determining, based on the model outputs, a consistency metric, the consistency metric indicating whether a quantity of feature sets in the combinations is sufficient to produce accurate output from the diagnostic machine learning model. Selectively ending the diagnostic testing with the patient based on a value of the consistency metric. Other implementations of this aspect include corresponding systems, apparatus, and computer programs, configured to perform the actions of the methods, encoded on computer storage devices.

These and other implementations can each optionally include one or more of the following features.

In some implementations, determining the consistency metric includes computing a variance of the model outputs.

In some implementations, selectively ending the diagnostic testing with the patient includes, in response to determining that the consistency metric is within a threshold value: causing a content presentation system to stop presenting trial content to the patient, and providing, for display on a user computing device, data indicating a diagnosis based on output data from the diagnostic machine learning model.

In some implementations, iteratively providing the different combinations of the feature sets as input data to the diagnostic machine learning model includes arranging a plurality of features sets into subsets that each include less than all of the plurality of feature sets.

In some implementations, selectively ending the diagnostic testing with the patient includes, in response to determining that the consistency metric is not within a threshold value: obtaining additional feature sets of additional diagnostic trials performed with the patient, iteratively providing new combinations of feature sets as input data to a diagnostic machine learning model to obtain new model outputs, and determining, based on the new model outputs, a new consistency metric, the new consistency metric indicating whether a new quantity of feature sets in the new combinations is sufficient to produce accurate output from the diagnostic machine learning model. In some implementations, some of the new combinations of feature sets include one or more of the additional feature sets and one or more of the feature sets.

In some implementations, the first number of diagnostic trials is a predetermined number of trials to produce a statistically relevant number of feature set combinations.

In some implementations, one or more feature sets that have a noise level above a threshold noise value are excluded from the combinations of the feature sets.

In some implementations, the consistency metric includes a distribution of consistency metrics.

In some implementations, selectively ending the diagnostic testing with the patient includes, in response to determining that a target percentage of the consistency metrics are within a threshold value: causing a content presentation system to stop presenting trial content to the patient, and providing, for display on a user computing device, data indicating a diagnosis based on output data from the diagnostic machine learning model.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1A depicts block diagram of an example diagnostic machine learning system in accordance with implementations of the present disclosure.

FIG. 1B depicts a block diagram that illustrates operations of a model tracker for the diagnostic machine learning system of FIG. 1A.

FIG. 2 depicts an example brainwave sensor system and stimulus presentation system according to implementations of the present disclosure.

FIG. 3 depicts a flowchart of an example process for analyzing the sufficiency of input for a machine learning model to produce accurate model output in accordance with implementations of the present disclosure.

FIG. 4 depicts a schematic diagram of a computer system that may be applied to any of the computer-implemented methods and other techniques described herein.

Like reference numbers and designations in the various drawings indicate like elements.

### DETAILED DESCRIPTION

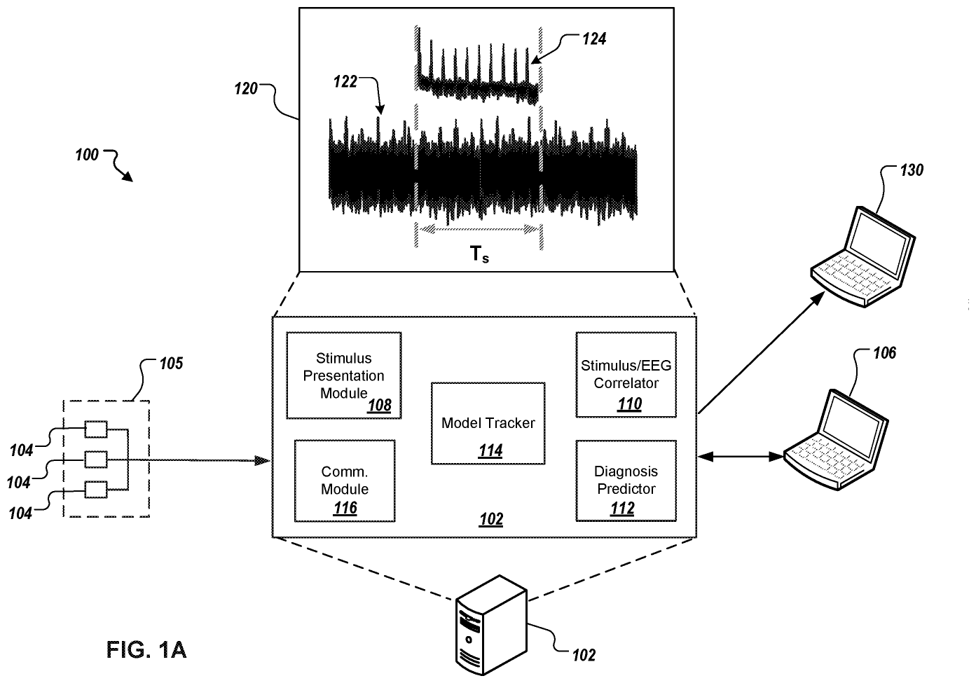

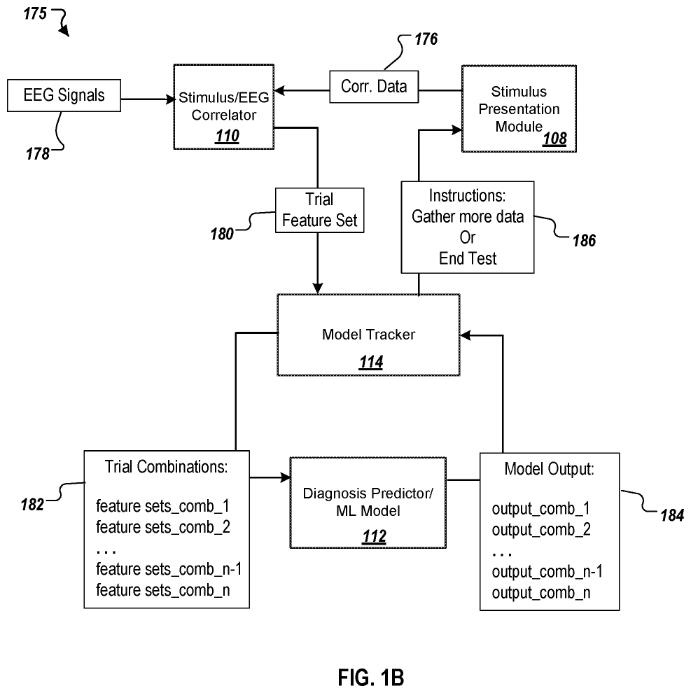

FIG. 1 depicts a block diagram of an example diagnostic system 100 (e.g., a machine learning based diagnostic system). The system includes a diagnosis module 102 configured to diagnose, e.g., mental health conditions in a patient such as depression or anxiety. The system includes a diagnosis module 102 which is in communication with brainwave sensors 104, a stimulus presentation system 106, and, optionally, a one or more user computing devices 130. The diagnosis module 102 can be implemented in hardware or software. For example, the diagnosis module 102 can be a hardware or a software module that is incorporated into a computing system such as a server system (e.g., a cloud-based server system), a desktop or laptop computer, or a mobile device (e.g., a tablet computer or smartphone). The diagnosis module 102 includes several sub-modules which are described in more detail below. As a whole, the diagnosis module 102 receives a patient's brainwave signals (e.g., EEG signals) from the brainwave sensors 104 while stimuli are presented to a patient. The diagnosis module 102 identifies brainwaves from particular brain systems that are generally responsive to specific media content presented as stimuli. In some examples, the content is presented to the patient in a series of trials while EEG data is measured from the patient during each of the trials. Considered together, the trials make up a diagnostic test used to obtain sufficient data from the patient in order to generate an accurate diagnosis of the patient's condition. The diagnosis system 100 can be, e.g., a psychiatric diagnosis system such as a system to diagnose or predict depression or anxiety in a patient.

While the present disclosure is described in the context of a mental health diagnostic system, it is understood that the techniques and processes described herein are applicable outside of this context. For example, the techniques and processes described herein may be applicable to other types of diagnostic machine learning systems including, but not limited to, medical diagnostic systems, computer software diagnostic (debugging) systems, computer hardware diagnostic systems, or quality assurance (e.g., in manufacturing) diagnostic systems.

The diagnosis module 102 uses a machine learning model to analyze identified brainwaves and predict the likelihood that the patient will experience, for example, depression within a predefined time in the future. For example, the diagnosis module 102 obtains EEG data of a patient's brainwaves while the patient is presented with content that is designed to trigger responses in brain systems related to, e.g., depression. During a diagnostic test, for example, a patient may be presented with content during several trials. Each trial can include content with stimuli designed to trigger responses in one particular brain system or multiple different brain systems. As one example a trial could include content with tasks for visual stimuli designed to stimulate only one particular brain system. As another example, a trial could include first content directing the patient to conduct a task that simulates one brain system and second content that includes visual stimuli that stimulates another brain system.

As described in more detail below, the content can include stimuli designed to trigger responses in brain systems such as the dopaminergic reward system and the amygdala. The diagnosis module 102 can correlate the timing of the content presentation with the brainwaves in both the temporal and spatial domains to identify brainwaves associated with the applicable brain system. The diagnosis module 102 analyzes the brainwave signals from one or more brain systems to identify stimulus response patterns that are indicative of a future risk of, e.g., depression. As discussed below, the diagnosis module 102 can employ a machine learning model trained on hundreds of clinical test data sets to predict a patient's future likelihood of experiencing depression. The diagnosis module 102 can provide a binary output or probabilistic output (e.g., a risk score) indicating the likelihood that the patient's will experience depression over a predefined period of time. For example, the diagnosis module 102 can predict the likelihood that the patient will become depressed within several months (e.g., 6 months, 9 months, 12 months, or 18 months) from the time that the patient's brainwaves are measured and analyzed. The diagnosis module 102 sends the output data to a computing device 130 associated with the patient's doctor (e.g., a psychiatrist), the doctor's office computer or mobile device.

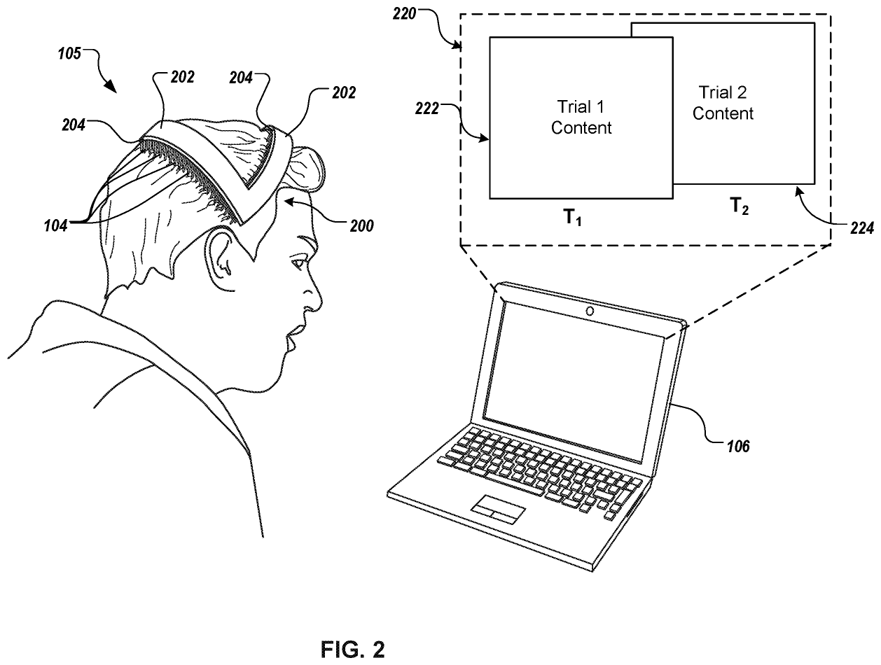

In general, any sensors capable of detecting brainwaves may be used. For example, the brainwave sensors 104 can be one or more individual electrodes (e.g., multiple EEG electrodes) that are connected to the diagnosis module 102 by wired connection. The brainwave sensors 104 can be part of a brainwave sensor system 105 that is in communication with the diagnosis module 102. A brainwave sensor system 105 can include multiple individual brainwave sensors 104 and computer hardware (e.g., processors and memory) to receive, process, and/or display data received from the brainwave sensors 104. Example brainwave sensor systems 105 can include, but are not limited to, EEG systems, a wearable brainwave detection device (e.g., as described below in reference to FIG. 2 below), a magnetoencephalography (MEG) system, and an Event-Related Optical Signal (EROS) system, sometimes also referred to as “Fast NIRS” (Near Infrared spectroscopy). A brainwave sensor system 105 can transmit brainwave data to the diagnosis module 102 through a wired or wireless connection.

FIG. 2 depicts an example brainwave sensor system 105 and stimulus presentation system 106. The sensor system 105 is a wearable device 200 which includes a pair of bands 202 that fit over a user's head. Specifically, the wearable device 200 includes one band which fits over the front of a user's head and the other band 202 which fits over the back of a user's head, securing the device 200 sufficiently to the user during operation. The bands 202 include a plurality of brainwave sensors 104. The sensors 104 can be, for example, electrodes configured to sense the user's brainwaves through the skin. For example, the electrodes can be non-invasive and configured to contact the user's scalp and sense the user's brainwaves through the scalp. In some implementations, the electrodes can be secured to the user's scalp by an adhesive.

The sensors 104 are distributed across the rear side 204 of each band 202. In some examples, the sensors 104 can be distributed across the bands 202 to form a comb-like structure. For example, the sensors 104 can be narrow pins distributed across the bands 202 such that a user can slide the bands 202 over their head allowing the sensors 104 to slide through the user's hair, like a comb, and contact the user's scalp. Furthermore, the comb-like structure sensors 104 distributed on the bands 202 may enable the device 200 to be retained in place on the user's head by the user's hair. In some implementations, the sensors 104 are retractable. For example, the sensors 104 can be retracted into the body of the bands 202.

In some examples, the sensors 104 are active sensors. For example, active sensors 104 are configured with amplification circuitry to amplify the EEG signals at the sensor head prior to transmitting the signals to a receiver in the diagnostic system 100 or the stimulus presentation system 105.

The stimulus presentation system 106 is configured to present content 220 to the patient for each diagnostic trial while the patient's brainwaves are measured during the diagnostic testing. For example, the stimulus presentation system 106 can be a multimedia device, such as a desktop computer, a laptop computer, a tablet computer, or another multimedia device. The content 220 is designed or selected to trigger responses in particular brain systems that are predictive of depression. For example, the content 220 can be selected to trigger responses in a patient's reward system (e.g., the dopaminergic system) or emotion system (e.g., the amygdala). The content 220 can include, but is not limited to, visual content such as images or video, audio content, or interactive content such as a game. For example, emotional content can be selected to measure the brain's response to the presentation of emotional stimuli. Emotional content can include the presentation of a series of positive images (e.g., a happy puppy), negative images (e.g., a dirty bathroom), and neutral images (e.g., a stapler). The emotional images can be presented randomly or in a pre-selected sequence. As another example, risk/reward content can be used to measure the brain's response to receiving a reward. Risk/reward content can include, but is not limited to, an interactive game where the patient choose one of two doors and can either win or lose a small amount of money (e.g., win=$1.00, lose=$0.50) depending on which door they choose. The order of wins and losses can be random. In some implementations, no content is presented, in order to measure the brain's resting state to obtain resting state brainwaves.

In some implementations, the content 220 is presented during multiple trials of a diagnostic test. Each trial can include separate trial content 222, 224. For example, during a first trial T1 first trial content 222 is presented to the patient. The first trail content 222 may include only one type of content (e.g., content designed to trigger only one brain system) or the first trail content may include multiple types of content. For example, the first trial content 222 can include the interactive game followed by emotional stimuli. The second trial content 224 (and content of subsequent trials) can include the same types of content as the first trail content 222, or different type(s) of content from that included in the first trial content 222.

In some implementations, the wearable device 200 is in communication with the stimulus presentation system 106, a laptop, tablet computer, desktop computer, smartphone, or brainwave data processing system. For example, the diagnosis module 102, or portions thereof, can be implemented as a software application on a computing device, a server system or stimulus presentation system 106. The wearable device 200 communicates brainwave data received from the sensors 104 to the computing device.

Referring again to FIG. 1, the diagnosis module 102 includes several sub-modules, each of which can be implemented in hardware or software. The diagnosis module 102 includes a stimulus presentation module 108, a stimulus/EEG correlator 110, a machine learning model 112, and a communication module 114. The diagnosis module 102 can be implemented as a software application executed by computing device 118. In some implementations, the sub-modules can be implemented on different computing devices. For example, one or both of the stimulus presentation module 108 and stimulus/EEG correlator 110 can be implemented on the stimulus presentation systems 106 with one or both of the stimulus/EEG correlator 110 and the machine learning model 112 being implemented on a server system (e.g., a cloud server system).

The communication module 114 provides a communication interface for the diagnosis module 102 with the brainwave sensors 104. The communication module 114 can be a wired communication (e.g., USB, Ethernet, fiber optic), wireless communication module (Bluetooth, ZigBee, WiFi, infrared (IR)). The communication module 114 can serve as an interface with other computing devices, the stimulus presentation system 106 and user computing devices 130. The communication module 114 can be used to communicate directly or indirectly, through a network, with the brainwave sensor system 105, the stimulus presentation system 106, user computing devices 130, or a combination thereof.

The stimulus presentation module 108 controls the presentation of stimulus content on the stimulus presentation system 106. The stimulus presentation module 108 can select content to trigger a response by particular brain systems in a patient. For example, the stimulus presentation module 108 can control the presentation of content configured to trigger responses in a dopaminergic system such as an interactive risk/reward game. As another example, the stimulus presentation module 108 can control the presentation of content configured to trigger responses in the amgydala system such as a sequence of emotionally positive, emotionally negative, and emotionally neutral emotional images or video. Moreover, the stimulus presentation module 108 can alternate between appropriate types of content to obtain samples of brain signals from each of one or more particular brain systems.

The stimulus presentation module 108 can send data related to the content presented on the stimulus presentation system 106 to the stimulus/EEG correlator 110. For example, the data can include the time the particular content was presented and the type of content. For example, the data can include timestamps indicating a start and stop time of when the content was presented and a label indicating the type of content. The label can indicate which brain system the content targeted. For example, the label can indicate that the presented content targeted a risk/reward system (e.g., the dopaminergic brain system) or an emotion system (e.g., the amygdala). The label can indicate a value of the content, whether the content was positive, negative, or neutral. For example, the label can indicate whether the content was positive emotional content, negative emotional content, or neutral emotional content. For example, for interactive content, the label can indicate whether the patient made a “winning” or a “losing” selection.

The stimulus/EEG correlator 110 identifies brainwave signals associated with particular brain systems within EEG data from the brainwave sensors 104. For example, the stimulus/EEG correlator 110 receives the EEG data from the brainwave sensors 104 and the content data from the stimulus presentation module 108. The stimulus/EEG correlator 110 can correlate the timing of the content presentation to the patient with the EEG data. That is, the stimulus/EEG correlator 110 can correlate the presentation of the stimulus content with the EEG data to identify brain activity in the EEG data that is responsive to the stimulus. Plot 120 provides an illustrative example. The stimulus/EEG correlator 110 uses the content data to identify EEG data 122 associated with a time period when the stimulus content was presented to the patient, a stimulus response period (Tₛ). The stimulus/EEG correlator 110 can identify the brainwaves associated with the particular brain system triggered by the content during the stimulus response period (Tₛ). For example, the stimulus/EEG correlator 110 can extract the brainwave data 124 associated with a brain system's response to the stimulus content from the EEG data 122. In some implementations, the stimulus/EEG correlator 110 can extract the brainwave data 124 in feature sets to be used as input to the machine learning model 112. In some implementations, the stimulus/EEG correlator 110 can tag the EEG data with the start and stop times of the stimulus. In some implementations, the tag can identify they type of content that was presented when the EEG data was measured.

The stimulus/EEG correlator 110 can send the brainwave signals associated with the particular brain systems to the model tracker 114. For example, the stimulus/EEG correlator 110 can send extracted brain wave signals that are associated with one or more brain systems as feature sets to the model tracker 114. In some examples, the stimulus/EEG correlator 110 can send tagged brainwave signals where the tags provide information including, but not limited to, an indication of brain system that the brainwaves are associated with, an indication of the type of content presented when the brainwaves were measured, and an indication of where in the brainwave signal the content presentation started.

In some implementations, values for parameters from the brainwave signals can, first, be extracted from the time domain brain wave signals and provided as input to the machine learning model. For example, values for a change in signal amplitude over specific time periods can be extracted from the brainwave signals and provides as model input. In some examples, the time periods can correspond to particular time intervals before, concurrent with, and/or after the stimulus content is presented to the patient. In some examples, time periods could also correspond to particular time intervals before, concurrent with, and/or after the patient makes a response to the stimulus. For example, values of the brainwave signals within a certain time period (e.g., within 1 second or less, 500 ms or less, 200 ms or less, 100 ms or less) of presentation of a stimulus to the patient during a trial can be extracted from the signals as trial feature sets for input to the machine learning model. More complex features of the brainwave signals can also be extracted and provided as input to the machine learning model. For example, frequency domain, time x frequency domain, regression coefficients, or principal or independent component factors can be provided to the model, instead of or in addition to, raw time domain brainwave signals.

The model tracker 114 iteratively tests the performance of the machine learning model 112 on input data (e.g., trial feature sets) as the data is obtained during diagnostic testing. The model tracker 114 receives feature sets of different trials during a diagnostic test from the stimulus/EEG correlator 110. The model tracker 114 stores the trial feature sets as they are received and arranges them in different combinations of feature sets to be used performance testing data for the machine learning model 112. The model tracker 114 iteratively provides different combinations of feature sets to machine learning model 112 to generate model output. The model output is fed back to the model tracker 114 where the model tracker 114 analyzes the model output to determine the consistency/reliability of the machine learning model's predictions. The model tracker 114 uses the analysis of the output data to evaluate whether additional data is needed to obtain reliable machine learning model outputs, or whether a sufficient quantity of input data (e.g., feature sets from diagnostic trials) has been obtained. Once sufficient input data has been obtained, the model tracker 114 can send a signal indicating that testing is complete to the stimulus presentation module 118.

In more detail, FIG. 1B depicts a block diagram 175 that illustrates operations of a model tracker 114 for the diagnostic machine learning system 100 of FIG. 1A. The model tracker 114 tracks performance of the machine learning model 112 based on the quantity, and in some implementations the quality (e.g., noisiness), of the input data. As discussed above, the stimulus/EEG correlator 110 is correlation data 176 from the stimulus presentation module 108 to identify and extract EEG signal features from EEG signals 178 measured from the patient. More specifically, for each trial of a diagnostic test with the patient, stimulus/EEG correlator 110 can extract EEG feature sets related to the given trial (e.g., trial feature sets 180). The stimulus/EEG correlator 110 passes the trial feature sets 180 to the model tracker 114.

The model tracker 114 stores the trial feature sets 180. Model tracker 114 arranges the received trial feature sets 180 into a plurality of different combinations of feature sets to be used as performance test data for evaluating the performance of the machine learning model 112. For example, once a predetermined number of trial feature sets 180 are available to the model tracker 114, the model tracker 114 begins the performance testing with the machine learning model 112. That is, in order to begin the performance testing the model tracker 114 will need enough trial feature sets 180 in order to start building combinations of input data for the machine learning model 112. When selecting trial feature set combinations, the model tracker 114 can employ a bootstrapping process to approximate a random sampling of model results. For example, the model tracker 114 can arrange the trial feature sets 1A0 into combinations that include less than all of the trial feature sets available at a given time. For example, once ten trial feature sets 180 are available, the model tracker 114 can arrange the trial feature sets 180 into forty-five unique combinations of eight different trial feature sets 180

$$( e . g . , nCr = \frac{n \vert}{r \vert ( n - r ) \vert} ; 10 C 8 = 45 combinations ) .$$

The model tracker 114 then provides each of the different feature set combinations 182 as input to the machine learning model 112. The machine learning model 112 processes each of the feature set combinations 182 to generate predictive inferences (e.g., model output 184) based on each respective combination of input. The model output 182 is fed back to the model tracker 114. Model tracker 114 then analyzes the model output 182 to determine a consistency metric that indicates whether the current quantity of feature sets is sufficient to produce an accurate output from the machine learning model 112. For example, the model tracker 114 can compute a statistical metric that indicates an consistency and/or reliability of the machine learning model 112 from processing, in this example, eight feature sets of input data. In some examples, the statistical metric computed by the model tracker 114 may be the variance of the model output 184 received from the machine learning model 112. Some implementations can use more complex metrics, such as kurtosis, Gaussian fit, or non-parametric distribution estimation.

The model tracker 114 determines whether the diagnostic test should be continued in order to gather additional input data for the machine learning model 112 based on the value of the determined consistency metric. For example, the model tracker 114 can determine whether the consistency metric is within a threshold value of consistency in order to consider the diagnostic test to be complete, and by extension, the model output 184 to be accurate. That is, a threshold value of consistency is used to indicate when the performance of the machine learning model 112 indicates that a sufficient amount of model input data has been gathered from the patient. For example, the model tracker 114 can compare the variance in the model output 184 to a predetermined threshold value. If, for example, the variance is within the threshold value (e.g., less than or equal to the threshold value) then sufficient input data has been gathered to produce accurate model outputs. In some examples, the threshold is determined by the computation of a non-parametric confidence interval of the test metric over the approximately random subset of combinations formed by the model tracker 114.

If the model tracker 114 determines that sufficient input data has been gathered, the model tracker 114 can send a signal 186 to the stimulus presentation module 108 instructing the stimulus presentation module 108 to end the diagnostic testing. If the model tracker 114 determines that the current data is not sufficient to produce accurate machine learning model output, then the model tracker 114 can indicate to the stimulus presentation module 108 that further trials are required.

The model tracker 114 repeats the above described process until the consistency metric indicates that a sufficient number of diagnostic trials have been completed to gather enough input data for the machine learning model 112 to generate accurate predictions. For example, as additional trials are completed the stimulus/EEG correlator 110 continues to send new trial feature sets 180 to the model tracker 114. The model tracker 114 continues to iteratively build larger combinations of feature sets using the new and stored feature sets and applying the feature set combinations to the machine learning model 112. For example, once twenty trial feature sets 180 are available, the model tracker 114 can arrange the trial feature sets 180 into one hundred and ninety unique combinations of eighteen different trial feature sets 180

$$( e . g . , nCr = \frac{n !}{r ! ( n - r ) !} ; 20 C 18 = 190 combinations ) .$$

As more data is gathered through performing additional diagnostic trials, the consistency of model output from machine learning model 112 will continue to improve. For example, this improved consistency of model output may improve the validity of the non-parametric confidence distribution built around the test metric.

In some implementations, the model tracker 114 determines whether the consistency metric indicates that a sufficient number of diagnostic trials have been completed by comparing the consistency metric to a threshold consistency value. For example, the model tracker 114 repeats the above described process until the consistency metric is within or equal to the bounds of a threshold value that is indicative of consistent model output data. For example, a consistency metric that decreases as the consistency of model output increases (e.g., variance) will indicate that a sufficient number of diagnostic trials have been completed when the consistency metric is less than or equal to the threshold consistency value. A consistency metric that increases as the consistency of model output increases will indicate that a sufficient number of diagnostic trials have been completed when the consistency metric is greater than or equal to the threshold consistency value.

In some implementations, the consistency metric includes a distribution of metrics. For example, the model tracker 114 can store multiple consistency metrics computed during different iterations of the model evaluation process to generate a distribution of consistency metrics from multiple trials of the model using increasingly larger combinations of input feature sets (e.g., a distribution of consistency metrics from multiple bootstrapping operations of the data). For example, the distribution of consistency metrics can be a distribution of the variances from different iterations of bootstrapping operations. The distribution of consistency metrics may indicate the improvements in model output consistency made over successive trials with increasingly more input data. In some implementations, the threshold consistency value can be related to a differential improvement in consistency metrics represented by the distribution of consistency metrics. For example, the threshold consistency value can represent a desired minimum difference between successive consistency values. In other words, decreasing improvements of model output consistency may indicate that the machine learning model is approaching or has reached its most consistent output using data from a given patient. For example, if the difference in variance between a trial using 50 input feature sets and a trial using 45 input feature sets shows minimal improvement, the model tracker 114 can end the diagnostic testing because the machine learning model has likely reached its most consistent output based on input data measured from that particular patient.

In some implementations, the model tracker 114 compares a distribution of consistency metrics to the threshold value to determine when sufficient input data has been obtained. For example, the model tracker 114 can use non-parametric confidence intervals to determine when diagnostic testing is complete, e.g., when sufficient input data has been obtained for the machine learning model. For example, the model tracker 114 can compare all or a subset of the consistency metrics in the distribution to the threshold consistency value to determine what percentage of the distribution is within the threshold value. Once a desired percentage of metrics in the distribution are within the threshold consistency value, the model tracker 114 can end the diagnostic testing. As a numerical example, the model tracker 114 may compare a distribution variances to a threshold value of 5. If only 20% of the variances are less than or equal to 5, the model tracker 114 continues to obtain input data from the patient. On the other hand, once 80% of the variances are less than or equal to 5, the model tracker 114 can end the diagnostic testing.

In some implementations, the model tracker 114 can remove noisy feature sets in order to improve the machine learning model's 112 performance. For example, as additional trial feature sets 180 are received from the stimulus/EEG correlator 110, the model tracker 114 can drop out noisy feature sets from consideration. For example, the model tracker 114 can remove model feature sets that are above a threshold signal-to-noise ratio. As the confidence distribution builds with more data fed to the model tracker 114 (and by extension to the machine learning model 112), the model tracker 114 can use outlier detection processes over that distribution to detect and remove noisy or unrepresentative data.

The machine learning model 112 determines a likelihood that the patient is experiencing or will experience a mental health condition, e.g., depression or anxiety. For example, the machine learning model 112 analyzes brainwave signals associated with one or more brain systems to determine the likelihood that the patient will experience a type of depression, major depressive disorder or post-partum depression, in the future. In some implementations, the machine learning model 112 analyzes resting state brainwaves in addition to brainwaves associated with one or more brain systems that are predictive of depression. In the context of measuring resting state brainwaves, since there may be no specific diagnostic trial associated with measuring resting state brainwaves (e.g., presentation of content to a patient) the features sets associated with resting state brainwaves are selected during different periods of time when the patient is at rest, e.g., times when no content that contains specific brain triggering stimulus is presented to the patient. In some implementations, the machine learning model 112 analyzes brainwave signals associated with one or more brain systems to determine the likelihood that the patient will experience anxiety in the future. For example, the machine learning model 112 can analyze brainwaves associated with brain systems that are predictive of anxiety.

The machine learning model 112 incorporates a machine learning model to identify patterns in the brainwaves associated with the particular brain systems that are predictive of future depression. For example, the machine learning model 112 can include a machine learning model that has been trained to receive model inputs, detection signal data, and to generate a predicted output, a prediction of the likelihood that the patient will experience depression in the future. In some implementations, the machine learning model is a deep learning model that employs multiple layers of models to generate an output for a received input. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a convolutional neural network. In some implementations, the machine learning model is an ensemble of models that may include all or a subset of the architectures described above.

In some implementations, the machine learning model can be a feedforward autoencoder neural network. For example, the machine learning model can be a three-layer autoencoder neural network. The machine learning model may include an input layer, a hidden layer, and an output layer. In some implementations, the neural network has no recurrent connections between layers. Each layer of the neural network may be fully connected to the next, there may be no pruning between the layers. The neural network may include an ADAM optimizer, or any other multi-dimensional optimizer, for training the network and computing updated layer weights. In some implementations, the neural network may apply a mathematical transformation, such as a convolutional transformation, to input data prior to feeding the input data to the network.

In some implementations, the machine learning model can be a supervised model. For example, for each input provided to the model during training, the machine learning model can be instructed as to what the correct output should be. The machine learning model can use batch training, training on a subset of examples before each adjustment, instead of the entire available set of examples. This may improve the efficiency of training the model and may improve the generalizability of the model. The machine learning model may use folded cross-validation. For example, some fraction (the “fold”) of the data available for training can be left out of training and used in a later testing phase to confirm how well the model generalizes. In some implementations, the machine learning model may be an unsupervised model. For example, the model may adjust itself based on mathematical distances between examples rather than based on feedback on its performance.

A machine learning model can be trained to recognize brainwave patterns from the dopaminergic system, the amygdala, resting state brainwaves, or a combination thereof, that indicate a patient's potential risk of one or more types of depression. For example, the machine learning model can correlate identified brainwaves from particular brain system(s) with patterns that are indicative of those leading to a type of depression such as major depressive disorder or post-partum depression. In some examples, the machine learning model can be trained on hundreds of clinical study data sets based on actual diagnoses of depression. The machine learning model can be trained to identify brainwave signal patterns from relevant brain systems that occur prior to the onset of depression. In some implementations, the machine learning model can refine the ability to predict depression from brainwaves associated brain systems such as those described herein. For example, the machine learning model can continue to be trained on data from actual diagnoses of previously monitored patients that either confirm or correct prior predictions of the model or on additional clinical trial data.

In some examples, the machine learning model 112 can provide a binary output, a yes or no indication of whether the patient is likely to experience depression or anxiety. In some examples, the machine learning model 112 provides a risk score indicating a likelihood that the patient will experience depression or anxiety (e.g., a score from 0-10 or a percentage indicating a probability that the patient will experience depression or anxiety). In some implementations, the depression predictor can output annotated brainwave graphs. For example, the annotated brainwave graphs can identify particular brainwave patterns that are indicative of future depression or anxiety. In some examples, the machine learning model 112 can provide a severity score indicating how severe the predicted depression or anxiety is likely to be.

In some implementations, the diagnosis module 102 sends output data indicating the patient's likelihood of experiencing depression to a user computing device 130. For example, the diagnosis module 102 can send the output of the machine learning model 112 to a user computing device 130 associated with the patient's doctor.

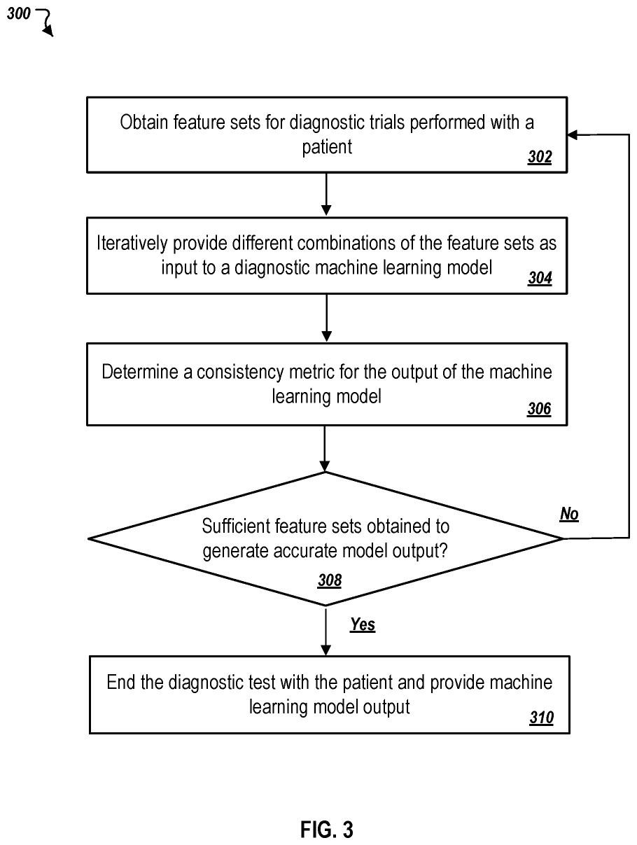

FIG. 3 depicts a flowchart of an example process 300 for analyzing the sufficiency of input for a machine learning model to produce accurate model output. In some implementations, the process 300 can be provided as one or more computer-executable programs executed using one or more computing devices. In some examples, the process 300 is executed by a system such as diagnosis module 102 of FIG. 1. In some implementations, all or portions of process 300 can be performed on a local computing device, a desktop computer, a laptop computer, or a tablet computer. In some implementations, all or portions of process 300 can be performed on a remote computing device, a server system, a cloud-based server system.

The system obtains feature sets for diagnostic trials performed with a patient (302). For example, the system measures the patient's brain waves while the patient is presented with one or more tasks or stimuli during each trial of a diagnostic test. For example, during each trial it patient may be presented with trial content (e.g., interactive tasks and/or stimuli) that is known to stimulate one or more desired human brain systems that may be indicative of a particular mental health condition such as depression or anxiety. The system can correlate the timing of when the patient is presented with the tasks were stimuli with the brain waves and extract brainwave feature sets from brain waves measured during each trial.

The system iteratively provides different combinations of feature sets as input to a diagnostic machine learning model (304). For example, during the diagnostic test, the system can arrange the already received trial feature sets into different combinations of input data for testing and performance of the machine learning model. The system iteratively provides the different combinations as input to the machine learning model in order to test whether the quantity of received data is sufficient to produce accurate and/or reliable output data from the model. For example, the system can arrange the already received trial feature sets into subsets of less than all of the trial feature sets received, where each subset includes a different combination of trial feature sets. In some implementations, the system does not begin the iterative model testing process until a predetermined number of feature sets have been obtained. For example, the system may delay the performance testing until a sufficient number of feature sets have been obtained to produce a statistically relevant number of feature set combinations.

System determines a consistency metric for the output of the machine learning model (306). For example, the system analyzes model outputs generated based on the supplied combinations of trial feature sets to determine the consistency of output generated by the machine learning model. In some examples, the system computes a variance of the model output data generated from the supplied combinations of feature sets. The consistency of results represented by the model output to a given quantity of input data (feature sets) can be representative of the performance of the machine learning model based on the quantity of input data at a given time during the diagnostic test. In other words, the more consistent the model output given a particular quantity of input data the more reliable and/or accurate model output can be considered. Regardless of how consistent the model is, a distribution of performance metrics can be built upon the multiple combinations of feature sets, which allows for the system to estimate the confidence that stable model output is within a given range. The more confidence and the narrower the range, the sooner data collection can stop. The necessary confidence and bandwidth could be set as parameters of the system (e.g., for some implementations an 80% confidence may be sufficient, while in other implementations a 95% confidence may be required).

The system selectively ends the diagnostic test based on analysis of the consistency metric. For example, if the consistency metric indicates that a sufficient number of feature sets have been obtained to generate accurate model output (308), the system ends the diagnostic test with the patient and provides the machine learning model output for presentation to a user such as a doctor or nurse (310). For example, the system can compare the consistency metric to a predetermined threshold value of consistency for the machine learning model. The threshold value may, for example, be different for different types of machine learning models and/or may change over time for a given machine learning model to account for improvements in the model as more data is analyzed over time.

If the value of the consistency metric is within the threshold value, the system will end the diagnostic test and presents the model output to a user. For example, the machine learning model can be configured to provide a binary output, e.g., a yes or no indication of whether the patient is likely to experience a particular mental health condition e.g., depression. In some examples, the machine learning model is configured to provide a risk score indicating a likelihood that the patient will experience depression (e.g., a score from 0-10). In some examples, the machine learning model is additionally configured to provide a severity score indicating how severe that depression is likely to be (e.g., 1=mild 2=moderate 3=severe). In some implementations, the machine learning model is configured to output annotated brainwave graphs. For example, the annotated brainwave graphs can identify particular brainwave patterns that are indicative of future depression. The system provides, for display on a user computing device, data indicating the likelihood that the patient will experience the determined mental health condition (e.g., depression) within the predefined period of time, and, optionally, how severe that depression is likely to be. For example, the system can provide the output of the machine learning model to a user computing device associated with the patient's doctor.

If the consistency metric does not indicate that a sufficient number of feature sets have been obtained to generate accurate model output (308), then the process 300 repeats steps (302)-(308); the system continues to gather more input data for the machine learning model and test the performance of the machine learning model. For example, the system continues to perform diagnostic test trials with the patient and obtain additional trial feature sets. The system uses the additional trial feature sets to expand the size and number of feature set combinations used to test the performance of the machine learning model. The system continues to analyze the model output produced by the expanding input feature set combinations until the system determines that a sufficient number of feature sets have been obtained generate accurate machine learning model output, e.g., as indicated by recomputing and reevaluating the consistency metric.

In some implementations, the consistency metric includes a distribution of metrics. For example, the system can store multiple consistency metrics computed during different iterations of the model evaluation process 400 to generate a distribution of consistency metrics from multiple trials of the model using increasingly larger combinations of input feature sets (e.g., a distribution of consistency metrics from multiple bootstrapping operations of the data). For example, the distribution of consistency metrics can be a distribution of the variances from different iterations of bootstrapping operations. The distribution of consistency metrics may indicate the improvements in model output consistency made over successive trials with increasingly more input data. In some implementations, the threshold consistency value can be related to a differential improvement in consistency metrics represented by the distribution of consistency metrics. For example, the threshold consistency value can represent a desired minimum difference between successive consistency values. In other words, decreasing improvements of model output consistency may indicate that the machine learning model is approaching or has reached its most consistent output using data from a given patient. For example, if the difference in variance between a trial using 50 input feature sets and a trial using 45 input feature sets shows minimal improvement, the system can end the diagnostic testing because the machine learning model has likely reached its most consistent output based on input data measured from that particular patient.

In some implementations, the system compares a the distribution of consistency metrics to the threshold value to determine when sufficient input data has been obtained. For example, the system can use non-parametric confidence intervals to determine when diagnostic testing is complete, e.g., when sufficient input data has been obtained for the machine learning model. For example, the system can compare all or a subset of the consistency metrics in the distribution to the threshold consistency value to determine what percentage of the distribution is within the threshold value. Once a desired percentage of metrics in the distribution are within the threshold consistency value, the system can end the diagnostic testing As a numerical example, the system may compare a distribution variances to a threshold value of 5. If only 20% of the variances are less than or equal to 5, the system continues to obtain input data from the patient. On the other hand, once 80% of the variances are less than or equal to 5, the system can end the diagnostic testing.

In addition, in some implementations, the system can choose trail data to reject based on a variance in the distribution that is an outlier from the rest of the distribution.

Further to the descriptions above, a patient may be provided with controls allowing the user to make an election as to both if and when systems, programs, or features described herein may enable collection of user information. In addition, certain data may be treated in one or more ways before it is stored or used, so that personally identifiable information is removed. For example, a patient's identity may be treated so that no personally identifiable information can be determined for the patient, or a patient's test data and/or diagnosis cannot be identified as being associated with the patient. Thus, the patient may have control over what information is collected about the patient and how that information is used.

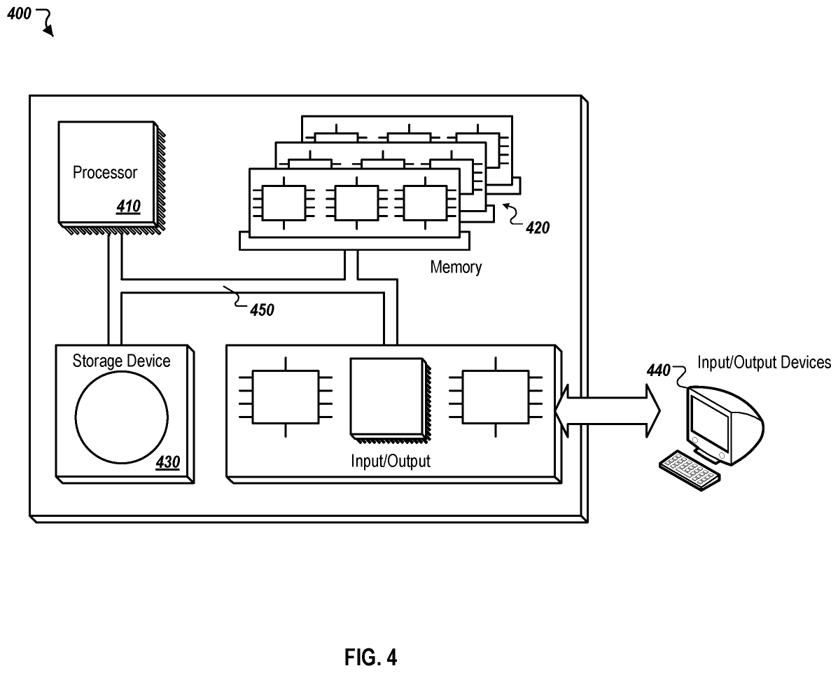

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

While the present disclosure is described in the context of a psychiatric diagnostic system, it is understood that the techniques and processes described herein are applicable outside of this context. For example, the techniques and processes described herein may be applicable to other types of diagnostic machine learning systems including, but not limited to, medical diagnostic systems, computer software diagnostic (debugging) systems, computer hardware diagnostic systems, or quality assurance (e.g., in manufacturing) diagnostic systems.

1. An input analysis system for a diagnostic electroencephalogram (EEG) system, comprising:
one or more processors;
one or more tangible, non-transitory media operably connectable to the one or more processors and storing instructions that, when executed, cause the one or more processors to perform operations comprising:
obtaining feature sets for a first number of diagnostic trials performed with a patient for diagnostic testing, wherein each feature set comprises one or more features of EEG signals measured from the patient while the patient is presented with trial content known to stimulate one or more desired human brain systems;
iteratively providing different combinations of the feature sets as input data to a diagnostic machine learning model to obtain model outputs, each model output corresponding to a particular one of the combinations;
determining, based on the model outputs, a consistency metric, the consistency metric indicating whether a quantity of feature sets in the combinations is sufficient to produce accurate output from the diagnostic machine learning model; and
selectively ending the diagnostic testing with the patient based on a value of the consistency metric.

2. The system of claim 1, wherein determining the consistency metric comprises computing a variance of the model outputs.

3. The system of claim 1, wherein selectively ending the diagnostic testing with the patient comprises, in response to determining that the consistency metric is within a threshold value:
causing a content presentation system to stop presenting trial content to the patient; and
providing, for display on a user computing device, data indicating a diagnosis based on output data from the diagnostic machine learning model.

4. The system of claim 1, wherein iteratively providing the different combinations of the feature sets as input data to the diagnostic machine learning model comprises arranging a plurality of features sets into subsets that each include less than all of the plurality of feature sets.

5. The system of claim 1, wherein selectively ending the diagnostic testing with the patient comprises, in response to determining that the consistency metric is not within a threshold value:
obtaining additional feature sets of additional diagnostic trials performed with the patient;
iteratively providing new combinations of feature sets as input data to a diagnostic machine learning model to obtain new model outputs; and
determining, based on the new model outputs, a new consistency metric, the new consistency metric indicating whether a new quantity of feature sets in the new combinations is sufficient to produce accurate output from the diagnostic machine learning model.

6. The system of claim 5, wherein some of the new combinations of feature sets include one or more of the additional feature sets and one or more of the feature sets.

7. The system of claim 1, wherein the first number of diagnostic trials is a predetermined number of trials to produce a statistically relevant number of feature set combinations.

8. The system of claim 1, wherein one or more feature sets that have a noise level above a threshold noise value are excluded from the combinations of the feature sets.

9. A computer-implemented input analysis method for calibrating a diagnostic system, the method executed by one or more processors and comprising:
obtaining, by the one or more processors, feature sets for a first number of diagnostic trials performed with a patient for diagnostic testing, wherein each feature set comprises one or more features of electroencephalogram (EEG) signals measured from the patient while the patient is presented with trial content known to stimulate one or more desired human brain systems;
iteratively providing, by the one or more processors, different combinations of the feature sets as input data to a diagnostic machine learning model to obtain model outputs, each model output corresponding to a particular one of the combinations;
determining, by the one or more processors and based on the model outputs, a consistency metric, the consistency metric indicating whether a quantity of feature sets in the combinations is sufficient to produce accurate output from the diagnostic machine learning model; and
selectively ending the diagnostic testing with the patient based on a value of the consistency metric.

10. The method of claim 9, wherein determining the consistency metric comprises computing a variance of the model outputs.

11. The method of claim 9, wherein selectively ending the diagnostic testing with the patient comprises, in response to determining that the consistency metric is within a threshold value:
causing a content presentation system to stop presenting trial content to the patient; and
providing, for display on a user computing device, data indicating a diagnosis based on output data from the diagnostic machine learning model.

12. The method of claim 9, wherein iteratively providing the different combinations of the feature sets as input data to the diagnostic machine learning model comprises arranging a plurality of features sets into subsets that each include less than all of the plurality of feature sets.

13. The method of claim 9, wherein selectively ending the diagnostic testing with the patient comprises, in response to determining that the consistency metric is not within a threshold value:
obtaining additional feature sets of additional diagnostic trials performed with the patient;
iteratively providing new combinations of feature sets as input data to a diagnostic machine learning model to obtain new model outputs; and
determining, based on the new model outputs, a new consistency metric, the new consistency metric indicating whether a new quantity of feature sets in the new combinations is sufficient to produce accurate output from the diagnostic machine learning model.

14. The method of claim 13, wherein some of the new combinations of feature sets include one or more of the additional feature sets and one or more of the feature sets.

15. The method of claim 9, wherein the first number of diagnostic trials is a predetermined number of trials to produce a statistically relevant number of feature set combinations.

16. The method of claim 9, wherein one or more feature sets that have a noise level above a threshold noise value are excluded from the combinations of the feature sets.

17. The method of claim 9, wherein the consistency metric comprises a distribution of consistency metrics.

18. The method of claim 17, wherein selectively ending the diagnostic testing with the patient comprises, in response to determining that a target percentage of the consistency metrics are within a threshold value:
causing a content presentation system to stop presenting trial content to the patient; and
providing, for display on a user computing device, data indicating a diagnosis based on output data from the diagnostic machine learning model.

19. A non-transitory computer readable storage medium storing instructions that, when executed by at least one processor, cause the at least one processor to perform operations comprising:
obtaining feature sets for a first number of diagnostic trials performed with a patient for diagnostic testing, wherein each feature set comprises one or more features of electroencephalogram (EEG) signals measured from the patient while the patient is presented with trial content known to stimulate one or more desired human brain systems;
iteratively providing different combinations of the feature sets as input data to a diagnostic machine learning model to obtain model outputs, each model output corresponding to a particular one of the combinations;
determining, based on the model outputs, a consistency metric, the consistency metric indicating whether a quantity of feature sets in the combinations is sufficient to produce accurate output from the diagnostic machine learning model; and
selectively ending the diagnostic testing with the patient based on a value of the consistency metric.

20. The medium of claim 19, wherein selectively ending the diagnostic testing with the patient comprises, in response to determining that the consistency metric is not within a threshold value:
obtaining additional feature sets of additional diagnostic trials performed with the patient;
iteratively providing new combinations of feature sets as input data to a diagnostic machine learning model to obtain new model outputs; and
determining, based on the new model outputs, a new consistency metric, the new consistency metric indicating whether a new quantity of feature sets in the new combinations is sufficient to produce accurate output from the diagnostic machine learning model.
